(19) United States
(12) Patent Application Publication
(10) Pub. No.: US 2013/0281915 A1
(43) Pub. Date: Oct. 24, 2013

(54) VACUUM ENABLED DEVICE WITH MICRO-NEEDLE INSERT FOR ACNE TREATMENT

(71) Applicant: ROBERT S. ANDERSON, Livermore, CA (US)

(72) Inventor: ROBERT S. ANDERSON, Livermore, CA (US)

(73) Assignee: Theravant, Inc., Livermore, CA (US)

(21) Appl. No.: 13/454,391

(22) Filed: Apr. 24, 2012

(51) Int. Cl. A61M 37/00; A61M 1/00

(52) U.S. Cl. 604/21; 604/290; 604/313

(57) ABSTRACT

Methods and an apparatus for extracting an underlying sebum of a pilosebaceous unit for the treatment of acne or closed comedones is disclosed. The method includes placing a vacuum suction pressure device on a surface of a skin area to be treated for an acne condition, applying the vacuum suction pressure on the surface of the skin area to pull the skin area into an aperture opening of the vacuum suction pressure device and to extract an underlying sebum of the pilosebaceous unit, puncturing the surface of the skin area containing at least one pilosebaceous unit with at least one micro-needle insert unit recessed into the aperture opening of the vacuum suction pressure device to create a lesion on the pilosebaceous unit and applying a subsequent dose of vacuum suction pressure or an application of broad band light to the skin area to be treated.

### FIELD OF INVENTION

This disclosure relates generally to the field of medical devices for a treatment of acne.

### BACKGROUND

Acne is a widely prevalent condition affecting millions of adults and teenagers in America and other parts of the world. Acne is one of most common disorders treated by the medical profession. It is also commonly treated by self-treatment by those affected by it. Acne can be a very stressful condition to cope with for adults and teenagers alike. Those that are affected by acne may seek out a myriad of treatment options and be disappointed by the results. Treatment options may vary for different people and may include, but not be limited to, systemic and topical antibiotics, retinoids, isotretrinoin and benzyl peroxide. While many of these treatments may be effective if used for a sustained period of time, most available treatment options may not show immediate results. The lack of effective treatment options having immediate efficacy may be frustrating and stressful for patients who are already taxed with having a constantly recurring condition like acne. Furthermore, patients may end up spending large amounts of money on ineffective treatments or even risk self-treatment by dangerous and ineffective means.

### SUMMARY

Disclosed are a method, an apparatus and/or a system of removing the underlying sebum in a pilosebaceous unit for the treatment of acne or closed comedones.

In one aspect, the method includes placing a vacuum suction pressure device on a surface of a skin area to be treated for closed comedones, applying the vacuum suction pressure on the surface of the skin area to pull the skin area into an aperture opening of the vacuum suction pressure device and to extract an underlying sebum of the pilosebaceous unit; puncturing the surface of the skin area containing at least one pilosebaceous unit with at least one micro-needle insert unit recessed into the aperture opening of the vacuum suction pressure device to create a lesion on the pilosebaceous unit; and releasing the application of the vacuum suction pressure on the surface of the skin area such that the punctured skin is released from the aperture opening of the vacuum suction pressure device.

In another aspect, an apparatus is disclosed that includes a vacuum suction pressure device to enclose a surface of a skin area containing the at least one pilosebaceous unit on the surface of a skin area to be treated for closed comedones, to pull in the surface of the skin area into an aperture of the vacuum suction pressure device, to extract, by the application of the vacuum suction pressure, the sebum contents of the pilosebaceous unit, to release the application of the vacuum pressure after the surface of the skin has been pulled up into the aperture of the vacuum suction pressure device, to apply subsequent application(s) of the vacuum suction pressure on the surface of the skin area containing the at least one pilosebaceous unit. The apparatus also includes a micro-needle insert recessed into the aperture of the vacuum suction pressure device to puncture the surface of the skin area pulled into the aperture of the vacuum suction pressure device creating at least one lesion on the surface of the skin area.

In yet another aspect, a method is disclosed that includes placing a vacuum suction pressure device on a surface of a skin area to be treated for closed comedones, applying the vacuum suction pressure on the surface of the skin area to pull up the skin area into an aperture opening of the vacuum suction pressure device and to extract an underlying sebum of the pilosebaceous unit, puncturing the surface of the skin area containing at least one pilosebaceous unit with at least one micro-needle insert unit recessed into the aperture opening of the vacuum suction pressure device to create a lesion on the pilosebaceous unit, releasing the application of the vacuum suction pressure on the surface of the skin area such that the punctured skin is released from the aperture opening of the vacuum suction pressure device and applying a subsequent dose of the vacuum suction pressure through the vacuum suction pressure device not having the micro-needle insert recessed into the aperture of the opening on the surface of the skin area to extract a remaining underlying sebum of the pilosebaceous unit through the lesion on the pilosebaceous unit.

### BRIEF DESCRIPTION OF FIGURES

Example embodiments are illustrated by way of example and not limitation in the figures of the accompanying drawings, in which like references indicate similar elements and in which:

FIGS. 1A and 1B illustrate a process flow diagram of the workings of the needle insert equipped vacuum suction pressure device.

FIG. 2 illustrates a system diagram of the medical device.

FIG. 3 illustrates a top view, side view and perspective view of the needle insert.

FIG. 4 shows a perspective view of the needle insert.

FIG. 5 illustrates how a needle insert is snapped into the vacuum suction pressure device.

FIG. 6 illustrates a basic anatomy of a pilosebaceous unit.

Other features of the present embodiments will be apparent from the accompanying drawings and from the detailed description that follows.

### DETAILED DESCRIPTION

Disclosed are a method, an apparatus and/or a system of sucking an underlying sebum of a pilosebaceous unit. In the following description, for the purposes of explanation, numerous specific details are set forth in order to provide a thorough understanding of the various embodiments. It will be evident, however, to one skilled in the art that the various embodiments may be practiced without these specific details.

Detailed Background and Causes

Acne is a disease of the pilosebaceous unit and may affect teenagers and adults alike. Acne is one of the most common disorders treated by doctors and medical personnel, and is also frequently treated by patients afflicted with acne themselves, sometimes with disastrous results. An acne condition may be a cystic acne condition and/or may manifest itself as small comedones, blackheads, whiteheads, papules, pustules and deep cysts, among others. As of now, there are many available treatments for the condition including, but not limited to, systemic and topical antibiotics, retinoids, isotretinoin and benzyl peroxide. While many of these treatments may be effective when used for a sustained period of time, most of these treatments typically do not produce immediate results, which is often the goal of patients opting for these types of treatments.

All acne lesions may start with the formation of a microcomedone created by hyperproliferation of the follicular epithelium. At such an early stage, the body may initiate a set of inflammatory processes and begin to activate the vascular endothelium. This initial inflammatory event may have multiple causes. For example, changes in the concentration of the lipids in sebum may be one such cause. In one or more embodiments, sebum may refer to a semifluid secretion of the sebaceous glands. It may also refer to an oily secretion of the sebaceous gland that may act as a lubricant for the hair and the skin and may even provide protection against bacteria.

Peroxisome proliferator-activated receptor (PPAR) s are transnuclear receptors responsible for generating peroxisome, which in turn are responsible for the catabolism of a variety of fats inside the cell. Studies linking androgens with PPAR in sebocytes suggest the increased production of free fatty acids and peroxidative squalene, both inflammatory compounds of which may be a cause for the initial inflammatory event as discussed above. Other evidence may also point to androgens linking to PPAR in sebocytes with increased sebocyte differentiation and lipid generation. Another cause may be free fatty acids in the cytoplasm of sebocytes that may also bind to PPAR, which in turn may lead to an increase in inflammatory compounds. All of the foregoing may be causes for the initial inflammatory event, and there may be one or more of the foregoing causes for the initial inflammatory event. All acne lesions or lesions of the pilosebaceous unit necessarily start with the initial inflammatory event caused by any of all of the above causes.

Increase in stress may also be another cause of an acne breakout. Recent studies have shown that immunoreactive nerve fibers in close proximity to the sebaceous glands generate a neuropeptide called substance P that may further stimulate the germative sebocyte cells leading to an increase in the number of these cells and in their differentiation.

The anaerobic bacterium pAcne may also be a source of the initial inflammatory event. Although studies have shown that it is not necessary for pAcne to be present to start a mirocomedone, its presence contributes to an inflammatory response. In acne lesions, human (b) defensis-1 and -2 are upregulated and serve as a protective mechanism against pathogens such as bacteria. It may be reasonable to assume that pAcne bacteria is responsible for this increase in human (b) defensis-1 and -2. In addition, Toll-like Receptors (TLR) manifested on macrophages may be found in acne lesions. TLRs may identify molecular patterns on bacteria and their presence may be an indication that pAcne bacteria have precipitated an inflammatory response.

The presence of inflammatory cytokines in the pilosebaceous unit may create hyperkeratinization, or a production of excess keratin. This excess may lead to an increase in the adherence or bonding of dead skin cells together resulting in a blocking of the hair follicle. A small cap may be formed. The inflammatory cytokines may also be the cause the epidermal/dermal barrier between the infundibulum and dermis to be compromised. Components of sebum may spill out into the dermis increasing inflammation, which may be main cause of scarring of the acne tissue. The accumulation of sebum may also increase the food supply and improve the anaerobic environment for the pAcne bacteria leading to a surge in their population further exacerbating the problem.

Although the cause of the acne lesion may be multifactorial, one major factor is an increase in sebum in the infundibulum unit. Removing the sebum and/or follicular contents may be one sound method for reducing an occurrence of acne. By removing sebum/follicular contents, the level of pAcne population may be immediately reduced. The various inflammatory cytokines contained in the sebum may be extracted. The inflammatory free fatty acids and peroxisomes may be removed as well. It may also be important how the sebum is removed from the infundibulum unit. Since the dermal/epidermal barrier is compromised, any method that applies pressure of the sebum without a corresponding pressure on the dermis may force the sebum out of the infundibulum into the dermis. The key may be to apply pressure on the dermis, thereby squeezing the sebum out of the infundibulum unit on to the surface of the skin.

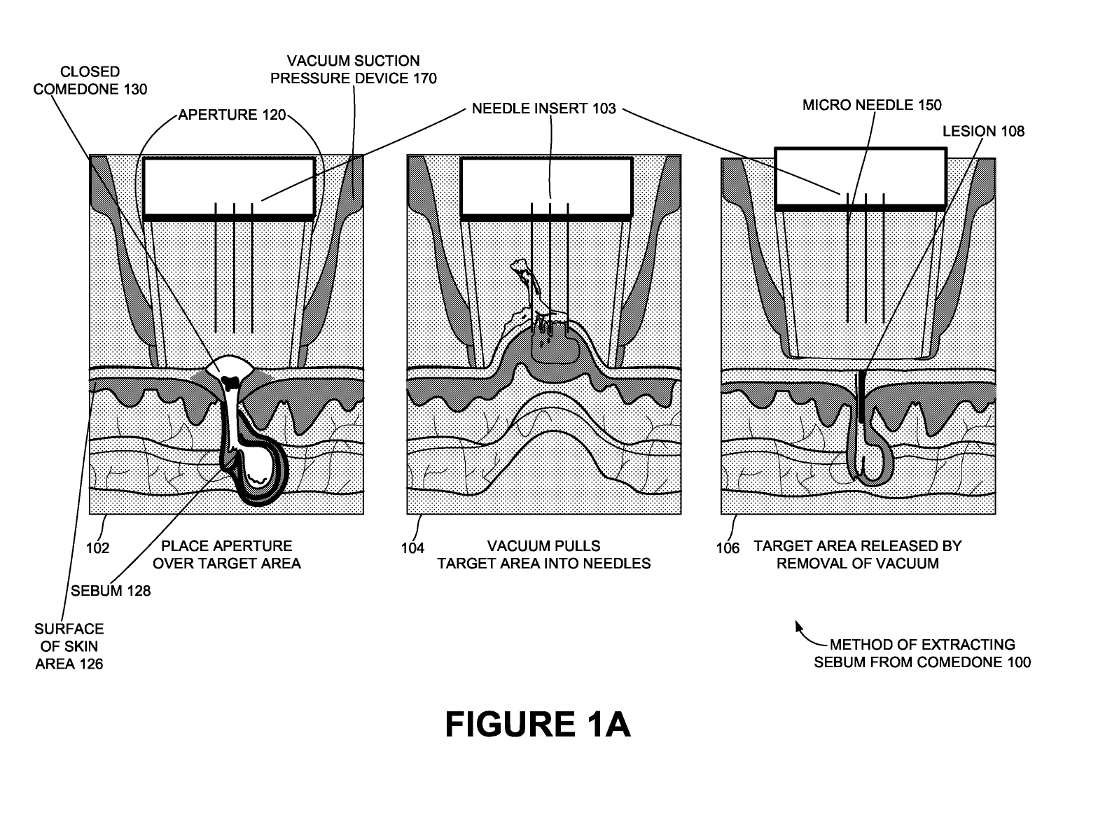

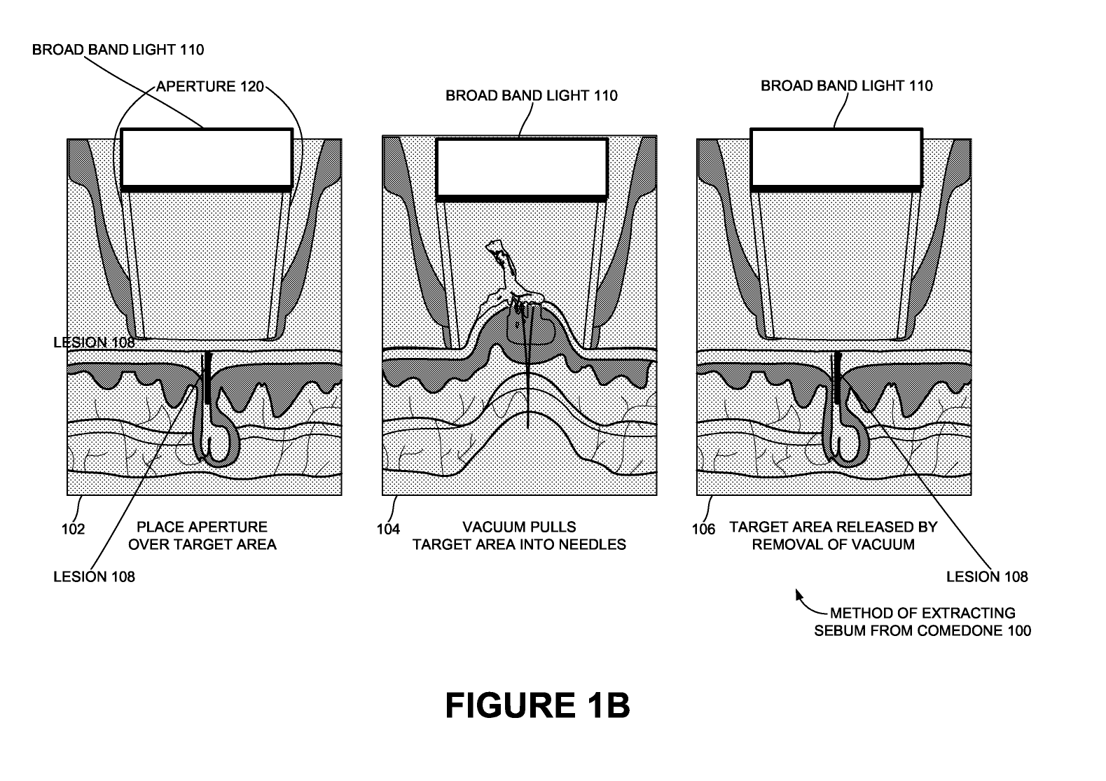

FIG. 1A is a process flow diagram of a needle insert equipped vacuum pressure device to assist in the removal of sebum from the pilosebaceous unit. FIG. 1 shows the micro-needle insert 103, the surface of the skin area 126, the sebum 128, the vacuum suction pressure device 150, the closed comedone 130, the micro-needle 150. FIG. 1B is a process flow diagram of the vacuum suction pressure device being applied to the skin without the needle insert and the aperture 120.

In one or more embodiments, the underlying sebum 128 of the pilosebaceous unit may be extracted by applying a vacuum pressure to the pilosebaceous unit such that the sebum 128 is sucked out of the unit and into the vacuum suction pressure device. However, if the opening of the follicle is closed, the underlying sebum 128 may not get extracted even if the uniform vacuum pressure is applied to the pilosebaceous unit.

In one or more embodiments, the vacuum suction pressure device 150 may be equipped with a needle insert 103. In one or more embodiments, the needle insert 103 may contain at least one needle. In one or more embodiments, the needle insert 103 may contain multiple needles. In one or more embodiments the needles of the needle insert 103 may be recessed such that the length of the needles is smaller than the length of a depth of the needle insert. In other words, the needles would only be able to touch a surface that is pulled into the depth of the needle insert. In one or more embodiments, the needle insert 103 may be designed with an area and/or length/width such that the needle insert 103 fits exactly into an aperture 120 of the vacuum suction pressure device 150. In one or more embodiments, the needle insert 103 snaps into the aperture 120 of the vacuum suction pressure device 150. In one or more embodiments, the top portion of the needle insert 103 may have holes such that the vacuum generated by the vacuum suction pressure device 150 is still able to function through the needles. In other words, the vacuum pressure generated by the device 150 is able to function and pull even with the presence of the needle insert.

In one or more embodiments, the vacuum suction pressure device 150 may be equipped with a negative pressure system and a positive pressure system that may enable the device 150 to generate a vacuum within the aperture 120 of the device 150. In one or more embodiments, the device 150 may be linked to a controller, processor, circuitry and other pulse electronics. In one or more embodiments, the device 150 may further contain at least one sensor, as will be explained later. In one or more embodiments, the device may be equipped with removable apertures that may fit into the main body of the device 150. For example, the device 150 may be able to generate a vacuum pressure of a great range, and the vacuum pressure to be applied may differ based on what part of the body is being treated. In one or more embodiments, the device 150 may be equipped with apertures of varying sizes. For example, when treating the face, a hand aperture of a smaller area may be used. When treating a larger area like the back, a larger aperture may be affixed to the device. As the size of the aperture changes, the size of the needle insert 103 may also change, in one or more embodiments. In one or more embodiments, needle insert 103 may closely mirror the size of the aperture 120.

The device 150 may be primarily a vacuum suction pressure device capable of generating a pressure of at least 1 psi to 15 psi, in one or more embodiments. In one or more embodiments, the pressure of the vacuum suction pressure device 150 may be anywhere from 1 psi to 15 psi. In one or more embodiments, the device may contain an aperture or an opening through which the vacuum pressure may be applied. In one or more embodiments, the aperture 120 may be large enough to cover at least one pilosebaceous unit. In one or more embodiments, the aperture 120 may cover an area of 1 mm×1 mm.

In one or more embodiments, a patient may be suffering from acute acne lesions on any part of his/her body. The patient may go to a treatment facility so that a medical personnel may be able to apply a treatment to the acne lesions on the patient. For example, the patient may have acne lesions on her cheek. In one or more embodiments, the medical personnel may first insert the needle insert 103 into the aperture 120 of the vacuum suction pressure device. In one or more embodiments, the medical personnel may then gently touch the aperture 120 of the device 150 containing the needle insert 103 to the treatment area—in this case the cheek—of the patient as shown in 102. In one or more embodiments, the medical personnel may turn the device 150 on, such that a vacuum pressure is applied through the aperture 120 of the device 150. In one or more embodiments, when the vacuum pressure is applied, the underlying skin that covers the pilosebaceous unit 104 may be pulled into the aperture 120 of the device 150 as shown in FIG. 1. In one or more embodiments, the needles of the needle insert 103 may puncture the skin and/or surface covering the pilosebaceous unit to create lesions on the surface of the pilosebaceous unit as shown in 104. In one or more embodiments, when the needle insert 103 contains multiple needles, multiple lesions may be created on the surface of the pilosebaceous unit.

In one or more embodiments, after applying the vacuum pressure of the device 150 with the needle insert 103 for a predetermined period of time, the medical personnel may turn off the vacuum pressure, such that the skin is released back from the aperture 120 and is no longer being pulled in to the aperture 120 containing the needle insert 103 as shown in 106. In one or more embodiments, the medical personnel may then remove the needle insert 103 from the aperture 120 of the medical device 150. In one or more embodiments, the medical personnel may go through an entire treatment area and may apply pressure uniformly throughout the treatment area through the needle insert 103 such that lesions are uniformly created on the surface of the skin.

As shown in FIG. 1B, in one or more embodiments, the medical personnel may then remove the needle insert 103 and go over the same treatment area and apply the vacuum pressure on at least one pilosebaceous unit. When the vacuum pressure is applied to the pilosebaceous unit after the needles have created lesions on the surface, the surface of the unit may once again be pulled into the aperture 120 of the device 150. In one or more embodiments, with the creation of the lesion 180 on the surface of the skin, the underlying sebum 128 of the pilosebaceous unit may be sucked into the aperture 120 of the device through the lesion 180 created by the needles of the needle insert. In one or more embodiments, the vacuum pressure may be applied for a minimum of 1 milliseconds and no more than 5 seconds. In one or more embodiments, sebum 128 may erupt through the lesion 180 and be extracted and pulled into the aperture 120 of the device 150 through the vacuum pressure generated by the device 150. After applying the vacuum pressure for the predetermined amount of time, the vacuum pressure may then be released. In one or more embodiments, the surface of the pilosebaceous unit may then be released and may be returned to its normal state.

In one or more embodiments, the medical personnel may then go over the same areas that contain the lesion 180, and either use the vacuum suction to extract any more remaining sebum 128, or in some cases, may use an application of broad band light 160 as will be described below. In such cases, the needle insert 103 may be removed, and the vacuum suction pressure device 150 may be placed on the surface of the skin area as shown in 108. The vacuum suction may then be applied such that the skin area is again pulled into the aperture 120 of the vacuum suction pressure device, but this time no lesion 180 is created because of the absence of the needle insert 103 as shown in 110. In one or more embodiments, the device may then generate a broad band light 160 such that the broad band light 160 is shone on the surface of skin. After the broad band light 160 is applied to the skin for the required period of time, the skin is released once again as shown in 112.

Although secondary to removing the sebum 128, the inflammation caused by acne may have a major vascular component. Very small capillaries, produced in response to the inflammatory signaling, may proliferate around and below the acne lesions. This increase in vasculature may be detected as redness on the surface of the skin. In one or more embodiments, an application of pulsed light of a proper wavelength and temporal duration may be an ideal energy source for removal of vessels. Since the hemoglobin and oxyhemoglobin in these capillaries have strong optical absorption in the green and yellow-orange portion of the spectrum, pulsed light in this spectral range may be ideal. Since the capillaries are very small, a short pulse duration in the millisecond range may be preferred.

In one or more embodiments, the device 150 may also be able to generate a combination of filtered broad band light 160 in addition to the application of vacuum pressure. In one or more embodiments, the broad band light 160 may be applied through a broad band light 160 insert that may also fit into the aperture 120 of the device 150. In another embodiment, the broad band light 160 may be applied contemporaneously to the vacuum suction. In another embodiment, the broad band light 160 may be applied after the vacuum suction. In another embodiment, the broad band light 160 may be applied simultaneously with the micro-needle insert. In another embodiment, the broad band light 160 may be applied through the aperture 120 of the vacuum suction pressure device 150 having the micro-needle insert recessed into the aperture 120. In other embodiments, the broad band light 160 may be applied through a separate aperture 120 in close proximity to the aperture 120 of the vacuum suction pressure device. In one or more embodiments, the broad band light 160 may be a light source generated through the aperture 120 of the device designed to shine broad band light 160 to the desired portion of the skin area. In one or more embodiments, the broad band light 160 may have a wide range, and may a wavelength of anywhere between 400 nm to 1800 nm.

In one or more embodiments, the broad band light 160 may be in the 400-1800 nm range. In one or more embodiments, the vacuum suction pressure may be around 3 psi. In one or more embodiments, when the surface of the pilosebaceous unit is pulled in through the vacuum generated by the device 150, the pulling of the pilosebaceous unit into the aperture 120 may also result in more light being delivered to the target area when compared to delivering light without the vacuum suction.

In one or more embodiments, in addition to the targeted heating of the dermis, the endogenous effect of the light may also activate porphyrins to destroy pAcnes bacteria and reduce sebum 128 production. In one or more embodiments, the pressure caused by the vacuum combined with the heat generated by the absorption of light may remove the follicular contents. In addition to the sebaceous contents, pAcnes may be removed both mechanically as well as thermally from the active acne lesion. When the vacuum and the light is applied to the pilosebaceous unit after the treatment areas have already been treated with the needle insert, the lesion 180 created on the surface creates a canal that may transport the sebum 128 out of the pilosebaceous unit and into the aperture 120 of the device 150 when the vacuum pressure is applied.

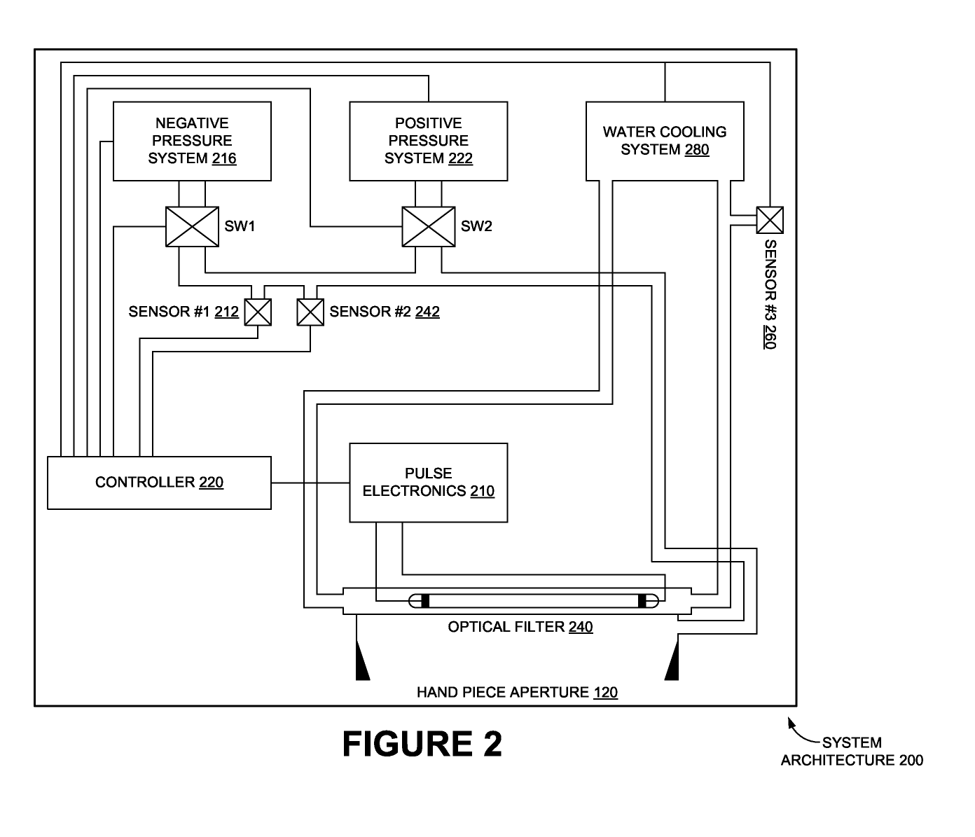

FIG. 2 illustrates the system of the device 150 further comprising a negative pressure system 216, a positive pressure system 222, a water cooling system 280, sensors 212 and 242, a controller 220, a pulse electronics module 210, an optical filter 240 and a hand piece aperture 120.

In one or more embodiments, the hand piece aperture may the aperture that may contain the micro-needle insert. In one or more embodiments, the hand piece aperture may be the aperture through which the vacuum suction is applied to the skin. In one or more embodiments, to generate the vacuum suction pressure at the aperture 120 of the vacuum suction pressure device, the system of the medical device may contain a negative pressure system and a positive pressure system. The pressure system, in combination, enables the vacuum suction pressure device 150 to achieve a vacuum suction pressure as low as 1 psi below atmospheric pressure, in one or more embodiments. In one or more embodiments, in the absence of any vacuum, atmospheric pressure may be about 15 psi. The pressure system, including the negative pressure system and the positive pressure system produce a vacuum such that the pressure of the system may go down to as low as 1 psi. The pressure system may be able to generate a pressure based on the requirements of the procedure. For example, a doctor or clinician may want to generate a weaker vacuum of say 7 psi. The system, with the negative pressure system and the positive pressure system and the sensors may be able to detect the pressure and apply the needed pressure through the aperture 120. The negative pressure system and the positive pressure system may be worked through a controller and a pulse electronics module that may control the workings of the pressure system and hold the electronics behind the workings of the device, in one or more embodiments. The water cooling system may help the system cool down if too much heat is generated by the device. The optical filter may filter out portions of the broadband light that may be generated through the aperture 120 of the system and not desired in the treatment.

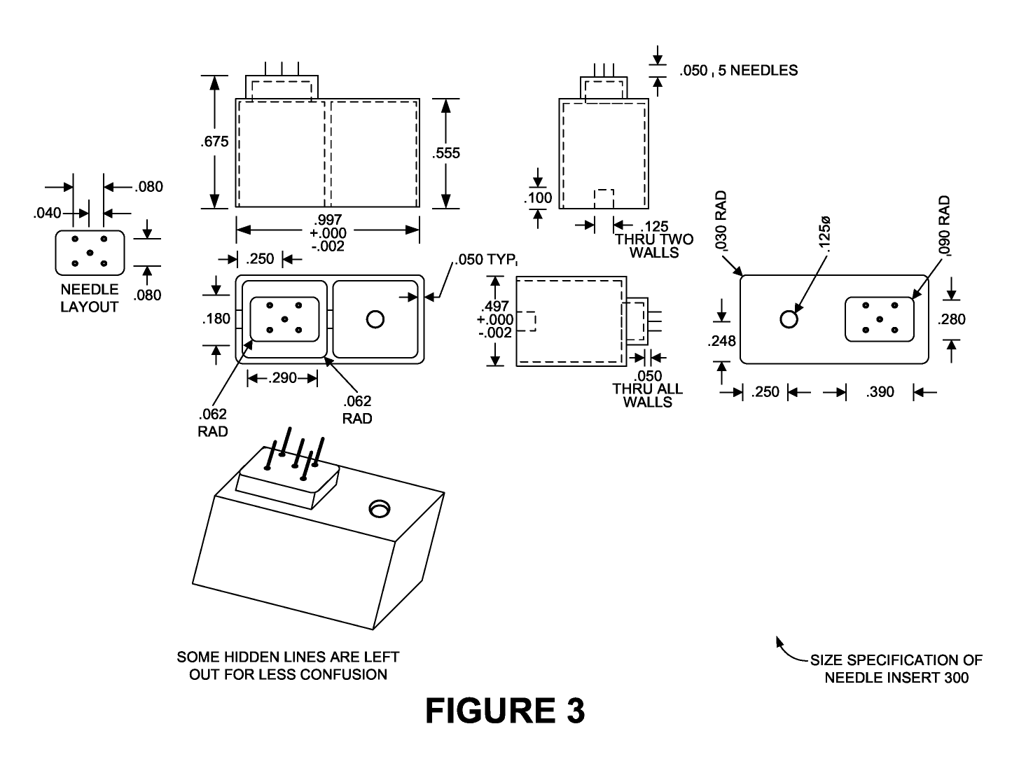

FIG. 3 illustrates the size specifications of the needle insert.

In one or more embodiments, the diameter of the micro needle may be anywhere between 100 microns to 400 microns. The needle insert 103 may have a size specification based on the size of the aperture 120. Some sample size specifications are illustrated in FIG. 3. The needle insert 103 may have a single needle in some embodiments. In other embodiments, the needle insert 103 may have multiple needles, as shown in FIG. 3.

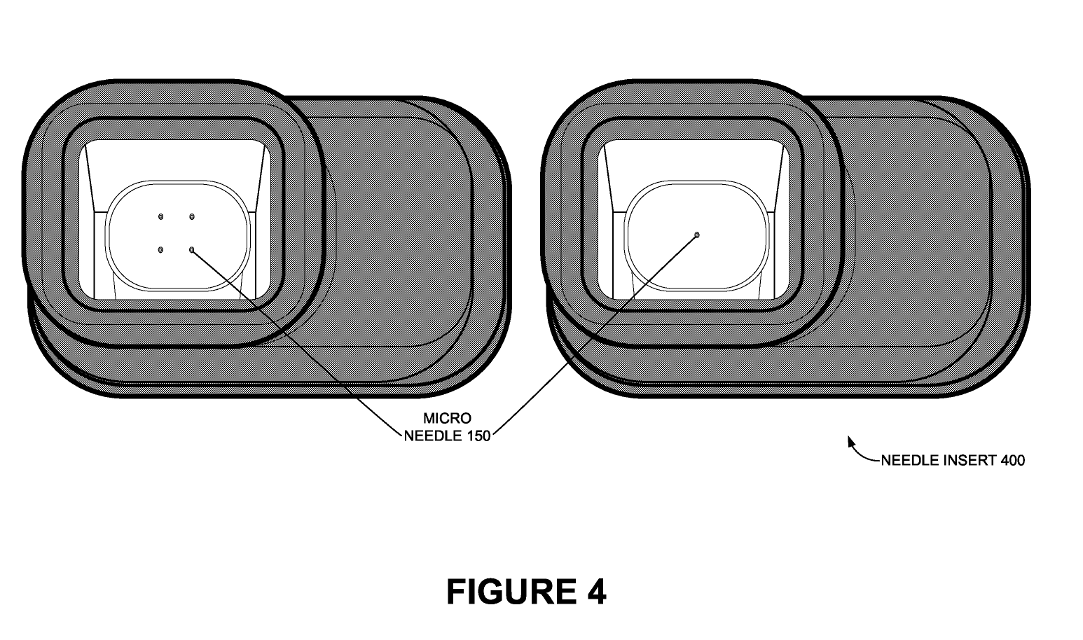

FIG. 4 shows a close up perspective view of the needle insert.

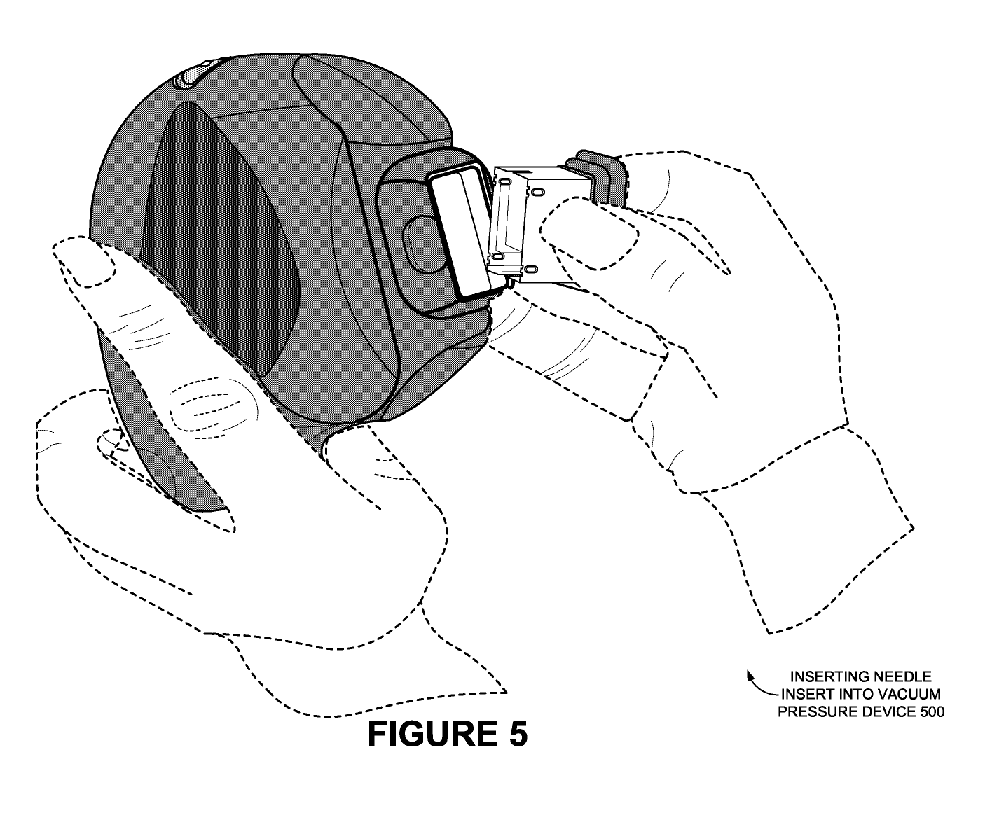

In one or more embodiments, the needle insert 103 shown in the Figure, may be inserted into the aperture 120 of the system, as shown in FIG. 2 and FIG. 1 and further in FIG. 5. In one or more embodiments, the size specification of the needle insert 103 may have to match the size specifications of the aperture 120 almost exactly such that the needle insert 103 fits into the aperture 120 snugly. In one or more embodiments, the needle insert 103 may have only one micro-needle or may have multiple micro-needles.

FIG. 5 shows the needle insert 103 being inserted into the aperture 120 of the vacuum suction pressure device 150. As mentioned above, the needle insert 103 may fit into the aperture 120 of the device to be then used on a desired skin area. The needle insert 103 may be easily removed after the first dose of vacuum suction pressure has been applied and the surface of the skin area 126 has been punctured. A subsequent dose of vacuum suction pressure may be applied to the same skin area, but this time after removing the needle insert.

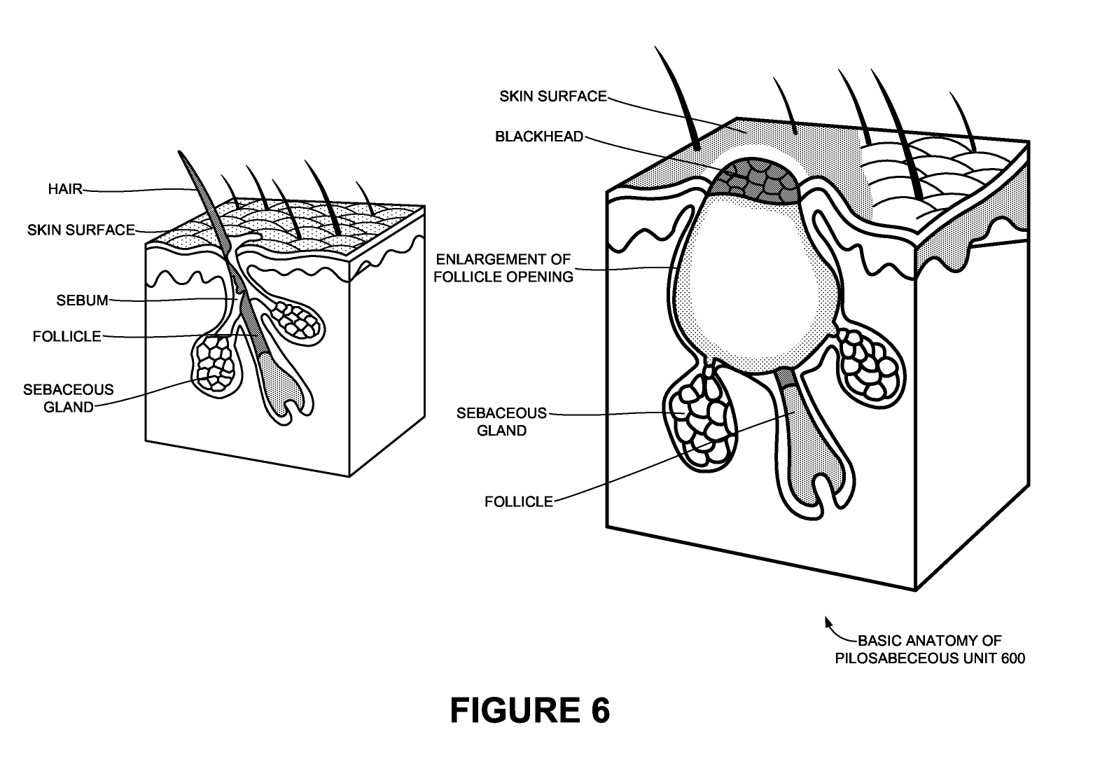

FIG. 6 illustrates a basic anatomy of a pilosebaceous unit.

As shown in the Figure, the skin area on top may have several hair follicles. There may be multiple pores on the skin surface, and there may be a blackhead on the surface of the skin. Below the blackhead, there may be an enlargement of the follicle opening cause by the blackhead itself. The sebaceous glands may be oil glands right below the opening, and may typically look like that shown in FIG. 6, without the enlargement of the follicle opening. The opening of the follicle and the sebaceous glands may have sebum 128. In an acne curing reducing procedure such as the one described in this application, a removal or reduction of sebum 128 may result in a removal of the blackhead, or any other acne condition.

Although the present embodiments have been described with reference to specific example embodiments, it will be evident that various modifications and changes may be made to these embodiments without departing from the broader spirit and scope of the various embodiments. For example, the various devices and modules described herein may be enabled and operated using hardware, firmware and software (e.g., embodied in a machine readable medium). For example, the various electrical structure and methods may be embodied using transistors, logic gates, and electrical circuits (e.g., application specific integrated (ASIC) circuitry and/or in digital signal processor (DSP) circuitry).

In addition, it will be appreciated that the various operations, processes, and methods disclosed herein may be embodied in a machine-readable medium and/or a machine accessible medium compatible with a data processing system (e.g., a computer devices), may be performed in any order (e.g., including using means for achieving the various operations). Accordingly, the specification and drawings are to be regarded in an illustrative rather than a restrictive sense.

1. A method comprising:
placing a vacuum suction pressure device on a surface of a skin area to be treated for an acne condition;
applying a vacuum suction pressure on the surface of the skin area to pull up the skin area into an aperture opening of the vacuum suction pressure device and to extract an underlying sebum of a pilosebaceous unit;
puncturing the surface of the skin area containing at least one pilosebaceous unit with at least one micro-needle insert unit recessed into the aperture opening of the vacuum suction pressure device to create a lesion on the pilosebaceous unit and to extract an underlying sebum of the pilosebaceous unit, wherein the micro-needle insert unit comprises at least one of a solid micro-needle and a plurality of solid micro-needles; and
releasing an application of the vacuum suction pressure on the surface of the skin area such that a punctured skin is released from the aperture opening of the vacuum suction pressure device.

2. The method of claim 1 further comprising:
applying a subsequent dose of the vacuum suction pressure through the vacuum suction pressure device not having the micro-needle insert recessed into the aperture of the opening on the surface of the skin area to extract a remaining underlying sebum of the pilosebaceous unit through the lesion on the pilosebaceous unit.

3. The method of claim 1 further comprising:
applying a broad band light, through at least one of the aperture of the vacuum suction pressure device having the micro-needle insert recessed into the aperture and an other aperture of the vacuum suction pressure device designated for the broad band light simultaneously when puncturing the surface of the skin area with the micro-needle insert recessed into the aperture of the vacuum suction pressure device for a period of at least 1 milliseconds and no more than 5 seconds.

4. The method of claim 1 further comprising:
applying a broad band light, through the aperture of the vacuum suction pressure device not having the micro-needle insert recessed into the aperture, for a period of at least 1 milliseconds and no more than 5 seconds to the surface of the skin area simultaneously when applying the subsequent dose of the vacuum suction pressure.

5. The method of claim 1 further comprising:
applying a broad band light, through the aperture of the vacuum suction pressure device not having the micro-needle insert recessed into the aperture, for a period of at least 1 milliseconds and no more than 5 seconds to the surface of the skin area, after the surface of the skin area has been punctured.

6. The method of claim 1 further comprising:
applying the broad band light, through the aperture of the vacuum suction pressure device not having the micro-needle insert recessed into the aperture, for a period of at least 1 milliseconds to the surface of the skin area after applying a subsequent dose of the vacuum suction pressure on the surface of the skin area.

7. The method of claim 1 further comprising:
pulling the skin area into the aperture of the vacuum suction pressure device to create a lesion of a depth of at least 0.5 mm below the surface of the skin area.

8. The method of claim 1 wherein a range of vacuum suction pressure applied on the skin area is between 1 psi to 15 psi relative to atmospheric pressure.

9. The method of claim 2 wherein a range of broad band light applied on the surface of the skin area is between 400 nm to 1800 nm.

10. The method of claim 1 wherein an area of the surface of the skin area to be treated is at least 1 mm×1 mm.

11. The method of claim 1 wherein the micro-needle insert recessed into the aperture of the vacuum suction pressure device is covered with a sponge material such that the sponge material is compressed when the surface of the skin area is pulled up into the aperture of the vacuum suction pressure device.

12. An apparatus comprising:
a vacuum suction pressure device:
to enclose a surface of a skin area containing at least one pilosebaceous unit on the surface of a skin area to be treated for closed comedones,
to pull in the surface of the skin area into an aperture of the vacuum suction pressure device,
to extract, by an application of a vacuum suction pressure, a sebum contents of a pilosebaceous unit,
to release the application of the vacuum suction pressure after the surface of the skin has been pulled up into the aperture of the vacuum suction pressure device,
to apply a subsequent application(s) of the vacuum suction pressure on the surface of the skin area containing at least one pilosebaceous unit; and
a micro-needle insert recessed into the aperture of the vacuum suction pressure device:
to puncture the surface of the skin area pulled into the aperture of the vacuum suction pressure device creating at least one lesion on the surface of the skin area, wherein the micro-needle insert comprises at least one of a solid micro-needle and a plurality of solid micro-needles.

13. The apparatus of claim 12 further comprising:
a broad band light generator coupled to the vacuum suction pressure device to simultaneously apply broad band light to the surface of the skin area with the subsequent application(s) of the vacuum suction pressure.

14. The apparatus of claim 12 wherein the surface of the skin area is pulled into the aperture of the vacuum suction pressure device to create a lesion of a depth of at least 0.5 mm below the surface of the skin area.

15. The apparatus of claim 12 wherein a range of vacuum suction pressure applied on the skin area is between 1 psi to 15 psi relative to atmospheric pressure

16. The apparatus of claim 12 wherein a range of broad band light applied on the surface of the skin area is between 400 nm to 1800 nm.

17. The apparatus of claim 12 wherein a range of diameter of the micro-needle is between 100 microns and 400 microns.

18. The apparatus of claim 12 wherein an area of the surface of the skin area to be treated is at least 1 mm×1 mm.

19. A method comprising:
placing a vacuum suction pressure device on a surface of a skin area to be treated for an acne condition;
applying a vacuum suction pressure on the surface of the skin area to pull up the skin area into an aperture opening of the vacuum suction pressure device and to extract an underlying sebum of a pilosebaceous unit;
puncturing the surface of the skin area containing at least one pilosebaceous unit with at least one micro-needle insert unit recessed into the aperture opening of the vacuum suction pressure device to create a lesion on the pilosebaceous unit, wherein the micro-needle insert unit comprises at least one of a solid micro-needle and a plurality of micro-needles;
releasing the application of the vacuum suction pressure on the surface of the skin area such that a punctured skin is released from the aperture opening of the vacuum suction pressure device; and
applying a subsequent dose of the vacuum suction pressure through the vacuum suction pressure device not having the micro-needle insert recessed into the aperture of the opening on the surface of the skin area to extract a remaining underlying sebum of the pilosebaceous unit through the lesion on the pilosebaceous unit.

20. The method of claim 19 further comprising:
applying a broad band light, through the aperture of the vacuum suction pressure device not having the micro-needle insert recessed into the aperture, for a period of at least 1 milliseconds and no more than 5 seconds to the surface of the skin area simultaneously when applying the subsequent dose of the vacuum suction pressure.

21. The method of claim 19 wherein a range of vacuum suction pressure applied on the skin area is between 1 psi to 15 psi relative to atmospheric pressure.
